(19) United States
(12) Patent Application Publication
(10) Pub. No.: US 2023/0213509 A1
(43) Pub. Date: Jul. 6, 2023

(54) LATERAL FLOW ASSAY DEVICE FOR DIAGNOSING TRAUMATIC BRAIN INJURY USING TIME-RESOLVED FLUORESCENCE ANALYSIS AND METHOD FOR DIAGNOSING TRAUMATIC BRAIN INJURY USING THE SAME

(71) Applicant: PRECISION BIOSENSOR INC., Daejeon (KR)

(72) Inventors: Jongeun KANG, Cheongju-si (KR); Kyu-Youn HWANG, Sejong-si (KR); Jong-Myeon PARK, Seongnam-si (KR); Hanshin KIM, Anyang-si (KR)

(21) Appl. No.: 17/433,699

(22) PCT Filed: Sep. 7, 2020
(86) PCT No.: PCT/KR2020/012035

§ 371 Date: Aug. 25, 2021

(30) Foreign Application Priority Data
Apr. 1, 2020 (KR) 10-2020-0039763

(51) Int. Cl. G01N 33/543; G01N 33/58; G01N 33/68

(52) U.S. Cl. CPC G01N 33/54386; G01N 33/582; G01N 33/6896; G01N 2800/28

(57) ABSTRACT

Provided is a lateral flow assay device capable of detecting a traumatic brain injury marker including a sample pad into which a blood sample containing a traumatic brain injury marker is injected, an adsorption pad including a probe which is mixed to the marker when the traumatic brain injury marker moves from the sample pad to form a traumatic brain injury marker complex, and a porous film which fluid-communicates with the adsorption pad and capillary-migrates the traumatic brain injury marker complex from the adsorption pad to a detection line, in which the probe includes a capture antibody consisting of an antibody labeled with a specific binding material specifically binding to the traumatic brain injury marker and a detector antibody consisting of an antibody labeled with a fluorescent material having a relatively long emission lifetime of 1 microsecond or more, and a mixture of at least two different kinds-origin antibodies is used as the antibody labeled with the specific binding material or the fluorescent material.

### TECHNICAL FIELD

The present invention relates to a lateral flow assay device for diagnosing traumatic brain injury using time-resolved fluorescence analysis and a method for diagnosing traumatic brain injury using the same. More particularly, the present invention relates to a lateral flow assay device for diagnosing traumatic brain injury using time-resolved fluorescence analysis and a method for diagnosing traumatic brain injury using the same capable of providing high sensitivity even if the blood concentration of a glial fibrillary acidic protein as a mild traumatic brain injury (mTBI) marker is low.

### BACKGROUND ART

Brain injury is proud of a globally high incidence, and has problems in that a large number of CT scans for brain injury, particularly mild traumatic brain injury (mTBI) is not helped to determine meaningful brain injury after performing a general computer tomography (CT) scan, radiation exposure is not only large, but also space, time, and costly limitations are large.

Recently, some studies have been reported by targeting an increase in need of field diagnosis capable of reducing unnecessary CT scan number and more rapidly leaving the hospital by screening all patients with mild traumatic brain injury (mTBI) through a simple blood test (Berger et al., 2007; Poli-de-Figueiredo et al., 2006).

However, until now, there is reported no high-reliable bio-assay that may appropriately classify patients.

In order to determine the severity of the brain injury, a bio-assay capable of accurately measuring a trace amount of biomarker in a blood sample with high sensitivity is required, and it should also be applicable to field diagnosis such as emergency rooms.

### DISCLOSURE

### Technical Problem

The present invention has been made in an effort to provide a lateral flow assay device for diagnosing traumatic brain injury using time-resolved fluorescence analysis and a method for diagnosing traumatic brain injury using the same capable of measuring the blood concentration of a glial fibrillary acidic protein (GFAP) using a lateral flow immune assay device with high sensitivity to detect or/and classify any brain-related traumatic severity and being useful for diagnosis and having high reliability.

### Technical Solution

An exemplary embodiment of the present invention provides a lateral flow assay device capable of detecting a traumatic brain injury marker including a sample pad into which a blood sample containing a traumatic brain injury marker is injected, an adsorption pad including a probe which is mixed to the marker when the traumatic brain injury marker moves from the sample pad to form a traumatic brain injury marker complex, and a porous film which fluid-communicates with the adsorption pad and capillary-migrates the traumatic brain injury marker complex from the adsorption pad to a detection line, in which the probe includes a capture antibody consisting of an antibody labeled with a specific binding material specifically binding to the traumatic brain injury marker and a detector antibody consisting of an antibody labeled with a fluorescent material having a relatively long emission lifetime of 1 microsecond or more, and a mixture of at least two different kinds-origin antibodies is used as the antibody labeled with the specific binding material or the fluorescent material.

### Advantageous Effects

According to an exemplary embodiment of the present invention, it is possible to measure at least GFAP concentration in the blood using a lateral flow immune assay device with high sensitivity to detect or/and classify any brain-related traumatic severity, particularly, mild traumatic brain injury and to provide a lateral flow assay device for field diagnosis of traumatic brain injury and a manufacturing method thereof useful for diagnosis of traumatic brain injury and with high reliability.

### DESCRIPTION OF THE DRAWINGS

FIG. 1 is a diagram illustrating a schematic diagram and an analysis principle of a lateral flow immune assay device for detecting a traumatic brain injury biomarker according to an exemplary embodiment of the present invention.

FIG. 2 is a flowchart for describing a method for diagnosing traumatic brain injury using a lateral flow assay device for field diagnosis of traumatic brain injury according to an exemplary embodiment of the present invention.

FIG. 3 is a graph of confirming non-specific reaction reduction in the case of using a mouse-origin antibody and in the case of using mouse and rabbit-origin antibodies together in various GFAP negative plasma samples.

FIG. 4 is a lateral flow assay sensor photograph showing a non-specific phenomenon according to a combination of mouse and rabbit-origin antibody pairs when introducing GFAP negative plasma.

FIG. 5 is a graph showing a difference in fluorescence signal for each GFAP concentration according to a combination of mouse and rabbit-origin antibody pairs.

FIG. 6 is a graph showing a difference in fluorescence signal for each GFAP concentration in plasma.

FIG. 7 is a graph showing an ELISA test result for each GFAP concentration in plasma.

### MODE FOR INVENTION

Hereinafter, a lateral flow assay device for field diagnosis of traumatic brain injury according to an exemplary embodiment of the present invention and a manufacturing method thereof will be described with reference to the accompanying drawings.

An exemplary embodiment of the present invention is provided to illustrate the present invention and is not intended to limit the present invention. In fact, it will be apparent to those skilled in the art that various changes and modifications may be made without departing from the spirit or scope of the present invention.

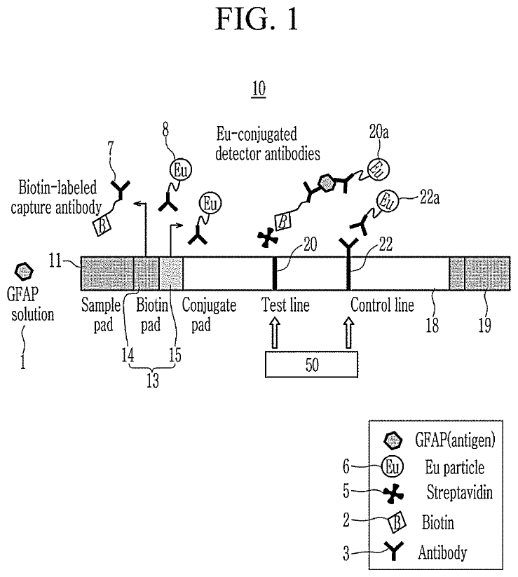

Referring to FIG. 1, a lateral flow assay device 10 for field diagnosis of traumatic brain injury according to an exemplary embodiment of the present invention includes a sample pad 11 into which a test sample containing an analyte (including a biomarker for diagnosing traumatic brain injury) is injected, an adsorption pad 13 including a probe which is mixed to the analyte moving from the sample pad 11 to form an analyte complex, a porous film 18 which fluid-communicates with the adsorption pad 13 and capillary-migrates the analyte complex from the adsorption pad 13 to a detection line 20, and an absorption pad 19 which is formed at an end of the porous film 18 to promote capillary action and fluid flow and accommodates waste after analysis, in which the sample pad 11, the adsorption pad 13, the porous film 18, and the absorption pad 19 may be supported by a rigid support.

Here, the “probe” may include a capture antibody 7 including an antibody 3 specifically binding to any one of traumatic brain injury markers included in the analyte, for example, glial fibrillary acidic protein (GFAP) (the probe is confused to mean a marker), S100B, UCH-L1, NSE, NeuN, CNPase, CAM-1, iNOS, MAP-1, MAP-2, SBDP145, SBDP120, III-tubulin, synaptic protein, neuroserpin, internexin, LC3, neurofacin, EAAT, DAT, nestin, cortin-1, CRMP, ICAM-1, ICAM-2, ICAM-5, VCAM-1, NCAM-1, NCAM-L1, NCAM-120, NCAM-140, NL-CAM, AL-CAM, or C-CAM1, and a detector antibody 8 including a fluorescent material 6 binding to the antibody 3.

In the lateral flow assay device 10 for field diagnosis of traumatic brain injury according to the exemplary embodiment of the present invention, the adsorption pad 13 may have first and second adsorption pads 14 and 15 sequentially provided by the probe. The first adsorption pad 14 may provide a specific binding material 2 forming the capture antibody 7 binding to the antibody 3 forming the analyte complex and the second adsorption pad 15 may provide the fluorescent material 6 forming the detector antibody 8 to provide a fluorescent marker to the analyte complex.

A capture material 5 capable of selectively binding to the binding material 2 included in the capture antibody 7 is immobilized on the detection line 20.

While the traumatic brain injury marker passes through the sample pad 11 and the adsorption pad 13, the capture antibody 7 labeled with the specific binding material 2 binds to the detector antibody 8 labeled with the fluorescent material 6 to form a specific analyte complex 20a and the specific analyte complex 20a is immobilized on the detection line 20 coated with the capture material 5 by interaction between the binding material 2 and the capture material 5.

In the lateral flow assay device 10 for field diagnosis of traumatic brain injury according to the exemplary embodiment of the present invention, the detection line 20 may include an antigen, a hapten, an antibody, a protein A, or G, avidin, streptavidin, a secondary antibody, and a biological capture material including a complex thereof.

In this specification, the biological capture material used streptavidin, and it is preferred to specifically bind to biotin which is the specific binding material 2 of the probe.

The capture material serves to provide a fixed binding site to the specific analyte complex 20a. In some examples, the analyte such as an antibody, an antigen and the like have two binding sites.

In the lateral flow assay device 10 for field diagnosis of traumatic brain injury according to the exemplary embodiment of the present invention, the adsorption pad 13 may include an antibody (detector antibody) labeled with the fluorescence without the capture antibody. In this case, on the detection line 20, the traumatic brain injury marker capture antibody is immobilized with a capture reagent to react with a traumatic brain injury marker-detector antibody complex.

When the analyte complex reaches the detection line 20, these binding sites are occupied with the specific binding material 2 of the complexed probe.

The detection line 20 is disposed in a line form in a direction substantially perpendicular to the flow of the sample, and the detection line 20 may indicate the presence of the analyte, but it is difficult to often measure the concentration of the analyte in the test sample by using only the detection line 20. Therefore, on the porous film 18, a control line 22 located on the downstream of the detection line 20 is provided.

The control line 22 may be provided with a capture material that may bind to any probe passing through the porous film 18.

In particular, any probes 22a that do not bind to the analyte are bound and fixed with the capture material of the control line 22 through the detection line 20.

The capture material used in the control line 22 may be different from the capture material 5 used in the detection line 20.

A probe fluorescent signal in the detection line 20 and the control line 22 may be measured using a time-resolved fluorescence tester 50.

The time-resolved fluorescence tester 50 is configured to simultaneously irradiate pulse excitation light to the detection line 20 and the control line 22, and may receive fluorescent signals emitted from the fluorescent material of the detection line 20 and the control line 22 at the same time.

The time-resolved fluorescence tester 50 may use one or more pulsed excitation sources and photodetectors that are linked with any other components such as an optical filter.

In this specification, the fluorescent material has a long emission lifetime of 1 microsecond or more and may use lanthanum chelates such as samarium (Sm(III)), dysprosium (Dy(III)), europium (Eu(III)), and terbium (Tb(III)) having both a relatively long emission lifetime and a large stoke migration so as to substantially remove background interference such as scattering light and self-fluorescence.

Therefore, the time-resolved fluorescence tester 50 may have a simple and inexpensive design. For example, the time-resolved fluorescence tester 50 may excite the fluorescent material using a light emitting diode (LED) and may also detect the fluorescence of the detection line 20 and the control line 22 without using an expensive component such as a monochrometer or a narrow emission bandwidth optical filter.

Meanwhile, since the glial fibrillary acidic protein (GAFP) which is one of markers for field diagnosis of mild traumatic brain injury (mTBI) has a low blood concentration, in order to increase the sensitivity of the lateral flow assay device 10 for diagnosing traumatic brain injury according to the exemplary embodiment of the present invention, the time-resolved fluorescence analysis was performed by varying a capture/detector antibody pair binding to the GAFP.

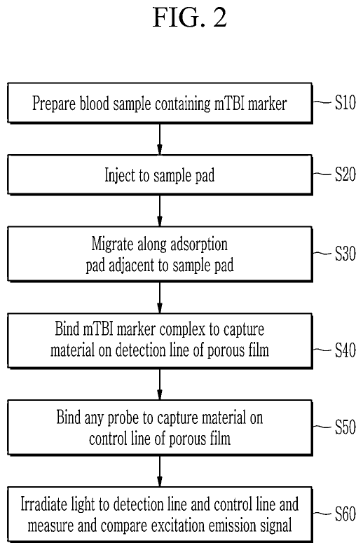

FIG. 2 is a flowchart for describing a method for diagnosing traumatic brain injury using a lateral flow assay device for diagnosing traumatic brain injury according to an exemplary embodiment of the present invention.

### EXPERIMENTAL EXAMPLE 1

A method for diagnosing traumatic brain injury using a lateral flow assay device for diagnosing traumatic brain injury according to an exemplary embodiment of the present invention is to detect a biomarker, GFAP in a sample using a lateral flow analysis method and a time-resolved fluorescence technique, and may include the steps of preparing a blood sample containing a mild traumatic brain injury (mTBI) marker (S10), injecting the blood sample containing the mild traumatic brain injury marker into a sample pad 11 (S20), forming a traumatic brain injury marker complex 20a consisting of a capture antibody 7 labeled with a specific binding material 2 and a detector antibody 8 labeled with a fluorescent material 6 while migrating the blood sample containing the traumatic brain injury marker along an adsorption pad 13 adjacent to the sample pad 11 with a capillary phenomenon (S30), migrating the traumatic brain injury marker complex 20a along a porous film 18 fluid-communicating with the adsorption pad 13 to bind to a capture material 5 on a detection line 20 of the porous film 18 (S40), binding any probe 22b not binding to the traumatic brain injury marker to a capture material of a control line 22 through the detection line 20 (S50), and measuring a concentration of the traumatic brain injury marker by irradiating light to the detection line 20 and the control line 22 from a time-resolved fluorescence tester 50 and comparing fluorescent signals of the detection line 20 and the control line 22 to diagnose the traumatic brain injury (S60).

A time-resolved fluorescence immune analysis method for detecting the presence or amount of GFAP in a test sample may include the steps of:

i) disposing a time-resolved fluorescence tester 50 by approaching a detection line 20, in which the time-resolved fluorescence tester 50 includes a pulse excitation source and a time-gated detector;

ii) emitting a detection signal from the fluorescent material binding to the GFAP by exciting the fluorescent material in the detection line 20 as the pulse excitation source; and

iii) analyzing the intensity of the detection signal with a time-gated detector.

First, various GFAP-negative plasma samples (GFAP level to 0 pg/mL) are injected to the lateral flow assay device 10 for field diagnosis of traumatic brain injury according to an exemplary embodiment of the present invention and then the fluorescence intensity was measured by using a time-resolved fluorescence measuring method.

As a result of measuring the corresponding samples through an ELISA kit (Creative Diagnostics, USA) sold on the market, it was confirmed that a very small optical signal OD at a buffer level was measured in all the samples.

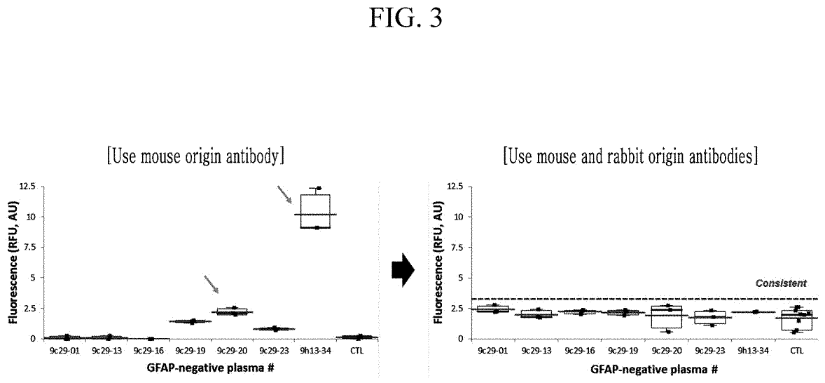

FIG. 3 is a graph of confirming non-specific reaction reduction in the case of using a mouse-origin antibody and in the case of using mouse and rabbit-origin antibodies at the same time in various GFAP negative plasma samples.

As shown in FIG. 3, in the case of using a mouse-origin antibody, non-specific characteristics are exhibited in a specific GFAP negative plasma sample, but in the case of using mouse and rabbit-origin antibodies at the same time, it may be seen that constant fluorescence intensities are shown in all the GFAP negative plasma samples and the non-specific reaction is reduced.

Accordingly, when in the adsorption pad 13, the specific binding material 2 uses biotin, the fluorescent material uses europium (Eu), and the antibody 3 forming the capture body 7 by binding to the biotin and the antibody 3 forming the detector antibody 8 by binding to the fluorescent material 6 use a mouse-origin antibody and a rabbit-origin antibody at the same time, it may be seen that it is preferred to exhibit the constant intensity and reduce the non-specific reaction.

This is because when only the rabbit-origin antibody is applied, it is difficult to implement the sensitivity, but when the rabbit-origin antibody and the mouse-origin antibody are simultaneously used, the sensitivity of a low concentration section may be secured.

The traumatic brain injury biomarker may use GFAP, S100B, UCH-L1, NSE, NeuN, CNPase, CAM-1, iNOS, MAP-1, MAP-2, SBDP145, SBDP120, III-tubulin, synaptic protein, neuroserpin, α-internexin, LC3, neurofacin, EAAT, DAT, nestin, corin-1, CRMP, ICAM-1, ICAM-2, ICAM-5, VCAM-1, NCAM-1, NCAM-L1, NCAM-120, NCAM-140, NL-CAM, AL-CAM, or C-CAM1.

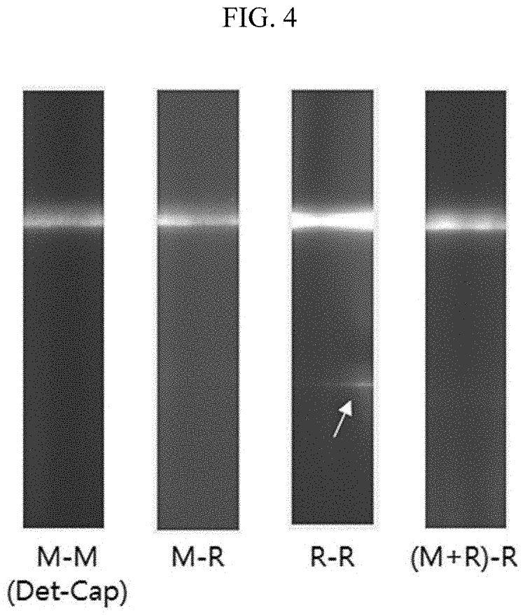

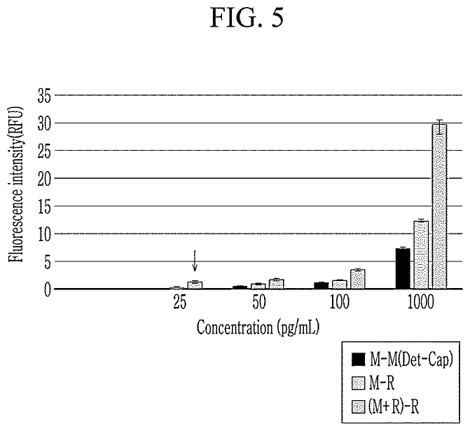

Then, the non-specific phenomenon according to an antibody origin was experimented with reference to FIGS. 4 and 5.

### EXPERIMENTAL EXAMPLE 2

A lateral flow sensor according to an antibody origin was manufactured to experiment a non-specific phenomenon and sensitivity according to the antibody origin. To prepare plasma samples with various GFAP concentrations, a GFAP material was purchased from Hytest Co., Ltd. and diluted continuously in GFAP negative plasma. It was confirmed that the concentration of the manufactured GFAP sample was accurate by using a commercial ELISA kit (Creative Diagnostics, USA).

FIG. 4 is a lateral flow assay sensor photograph showing a non-specific phenomenon according to a combination of mouse and rabbit-origin antibody pairs when introducing GFAP negative plasma and FIG. 5 is a graph showing a difference in fluorescence signal for each GFAP concentration according to a combination of mouse and rabbit-origin antibody pairs.

Here, a pair of the detector antibody 8 and the capture antibody 7 is represented by (Det-Cap), a mouse-origin antibody is represented by M, a rabbit-origin antibody is represented by R, and a mixture of mouse and rabbit-origin antibodies is represented by M+R.

Referring to FIG. 4, with respect of the pair (Det-Cap) of the detector antibody 8 and the capture antibody 7, in the case (R−R) of using only a rabbit-origin antibody, it may be confirmed that the non-specific signal is severely shown in a band form for the GFAP negative plasma.

Referring to FIG. 5, it may be seen that in a combination ((M+R)−R) of using a mixture of a mouse-origin antibody and a rabbit-origin antibody as the detector antibody 8 and using the rabbit-origin antibody as the capture antibody 7, a difference in fluorescence signal size according to a GFAP concentration and a fluorescence signal size even at a low concentration (25 pg/mL) are largest than a case (M−M) of using only the mouse-origin antibody as a pair of the detector antibody 8 and the capture antibody 7 or a case (M−R) of using the mouse-origin antibody and the rabbit-origin antibody as a pair of the detector antibody 8 and the capture antibody 7, respectively.

When comparing the case (M−M) of using only the mouse-origin antibody as the pair of the detector antibody 8 and the capture antibody 7 or the case (M−R) of using the mouse-origin antibody and the rabbit-origin antibody as the detector antibody 8 and the capture antibody 7, respectively, it may be seen that in the case (M−R) of using the mouse-origin antibody and the rabbit-origin antibody as the pair of the detector antibody 8 and the capture antibody 7, respectively, the fluorescence signal is excellent.

In the lateral flow assay device 10 for field diagnosis of traumatic brain injury according to the exemplary embodiment of the present invention, as illustrated in FIGS. 4 and 5, in the case (M−M) of using the mouse-origin antibody as the detector antibody 8 and the capture antibody 7, respectively, it may be seen that the sensitivity is lowered and there is a limitation in measurement of GFAP concentration to determine the brain injury severity.

Further, in the case of using a rabbit-origin antibody which is generally known that antibody-antigen reactivity is 10 to 100 times higher than the mouse-origin antibody, as the detector antibody 8 and the capture antibody 7, respectively, it may be seen that since non-specific bands are generated on a lateral flow sensor to distort a signal, it is not preferred to measure the concentration of the GFAP which is the traumatic brain injury biomarker.

Further, when the mouse-origin antibody is used as the detector antibody 8 and the rabbit-origin antibody is used as the capture antibody 7, it may be seen that non-specific bands are not generated and the sensitivity is partially increased as compared with when using the mouse-origin antibody alone, but it is not enough to determine a concentration level that may determine brain injury severity using GFAP.

Accordingly, in the case (M+R)−R) of using the mixture of the mouse-origin antibody and the rabbit-origin antibody as the detector antibody 8 and using the rabbit-origin antibody as the capture antibody 7, it may be seen that since the sufficient sensitivity may be implemented, it is preferred to measure the concentration of the traumatic brain injury biomarker.

### EXPERIMENTAL EXAMPLE 3

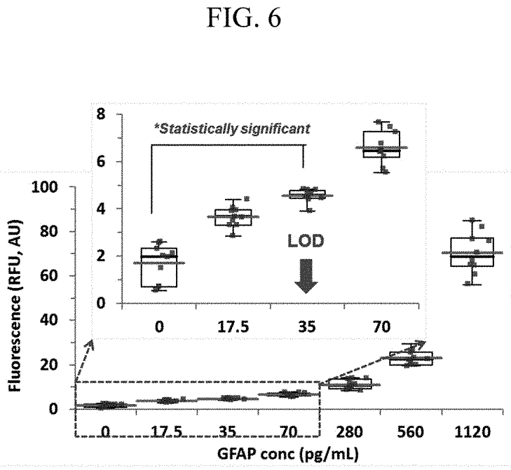

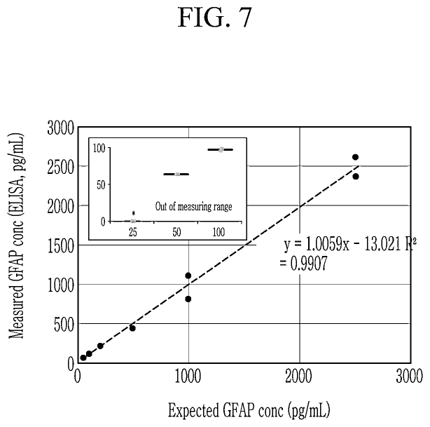

Hereinafter, GFAP measuring sensitivity using the lateral flow assay device for field diagnosis of traumatic brain injury according to the exemplary embodiment of the present invention will be described with reference to FIGS. 6 and 7.

FIG. 6 is a graph showing a result of measuring various GFAP concentrations using a lateral flow assay device for field diagnosis of traumatic brain injury according to an exemplary embodiment of the present invention and FIG. 7 is a graph showing a result of confirming the GFAP concentrations of FIG. 6 using an ELISA method.

In the lateral flow assay device for field diagnosis of traumatic brain injury according to the exemplary embodiment of the present invention, it may be seen that when in the content of a rabbit-origin antibody binding with europium nanoparticles, the detector antibody concentration is lowered to 0.1% to less than 2%, preferably 1% in an adsorption pad spray solution so that the non-specific bands are not generated, the sensitivity of a low concentration section between 20 pg/mL to 30 pg/mL is secured, but a signal deviation according to a concentration occurs.

As a result, when a mouse-origin antibody binding with europium nanoparticles is added to the adsorption pad spray solution at a concentration 3% to 12%, preferably 3% and the rabbit and mouse-origin antibodies are applied at the same time, the signal is secured even in the GFAP low-concentration section and the signal deviation according to the concentration is also improved.

As illustrated in FIG. 6, even in the GFAP concentration capable of determining the brain injury severity of less than about 100 pg/mL, preferably less than about 50 pg/mL, the sensitivity is provided to provide a lateral flow assay device for field diagnosis of traumatic brain injury with TRF-based high sensitivity.

In FIG. 7, it was confirmed through a commercial ELISA kit that the GFAP sample concentration used in the experiment was accurate. It was confirmed that the deviation between a manufacturing estimated concentration and an ELISA measurement concentration value was low, and the correlation was 0.99 or more.

Since the lateral flow assay device 10 for field diagnosis of traumatic brain injury according to the exemplary embodiment of the present invention exhibits the sensitivity even at a low-concentration biomarker GAFP concentration of 100 pg/mL, preferably 50 pg/mL for diagnosis of traumatic brain injury as described above, it may be seen that it is effective to diagnose the traumatic brain injury in combination with a Glasgow coma scale (GCS) (awake, language function, and exercise function).

### INDUSTRIAL APPLICABILITY

According to an exemplary embodiment of the present invention, it is possible to measure at least GFAP concentration in the blood using a lateral flow immune assay device with high sensitivity to detect or/and classify any brain-related traumatic severity, particularly, mild traumatic brain injury and to provide a lateral flow assay device for field diagnosis of traumatic brain injury and a manufacturing method thereof useful for diagnosis of traumatic brain injury and with high reliability.

1. A time-resolved fluorescence lateral flow assay device comprising:
in a lateral flow assay device capable of detecting a traumatic brain injury marker,
a sample pad into which a blood sample containing a traumatic brain injury marker is injected,
an adsorption pad including a probe which is mixed to the marker when the traumatic brain injury marker moves from the sample pad to form a traumatic brain injury marker complex, and
a porous film which fluid-communicates with the adsorption pad and capillary-migrates the traumatic brain injury marker complex from the adsorption pad to a detection line,
wherein the probe includes a capture antibody consisting of an antibody labeled with a specific binding material specifically binding to the traumatic brain injury marker and a detector antibody consisting of an antibody labeled with a fluorescent material having a relatively long emission lifetime of 1 microsecond or more.

2. The lateral flow assay device for traumatic brain injury using time-resolved fluorescence analysis of claim 1, wherein
the adsorption pad includes first and second adsorption pads sequentially provided by the probe, wherein the first adsorption pad provides a specific binding material forming the capture antibody and the second adsorption pad provides the fluorescent material forming the detector antibody.

3. The lateral flow assay device for traumatic brain injury using time-resolved fluorescence analysis of claim 1, wherein
a mixture of at least two different kinds-origin antibodies is used as the antibody labeled with the specific binding material or the fluorescent material.

4. The lateral flow assay device for traumatic brain injury using time-resolved fluorescence analysis of claim 1, wherein
the traumatic brain injury marker is a glial fibrillary acidic protein (GFAP), and any one of S100B, UCH-L1, NSE, NeuN, CNPase, CAM-1, iNOS, MAP-1, MAP-2, SBDP145, SBDP120, III-tubulin, synaptic protein, neuroserpin, internexin, LC3, neurofacin, EAAT, DAT, nestin, cortin-1, CRMP, ICAM-1, ICAM-2, ICAM-5, VCAM-1, NCAM-1, NCAM-L1, NCAM-120, NCAM-140, NL-CAM, AL-CAM, or C-CAM1.

5. The lateral flow assay device for traumatic brain injury using time-resolved fluorescence analysis of claim 4, wherein
biotin is used as the specific binding material, europium is used as the fluorescent material, and a mixture of a mouse-origin antibody and a rabbit-origin antibody is used as an antibody for the detector antibody.

6. The lateral flow assay device for traumatic brain injury using time-resolved fluorescence analysis of claim 5, wherein
in an antibody bound with europium particles, the content of the rabbit-origin antibody is at a concentration of 0.1% to less than 2%, preferably 1% in an adsorption pad spray solution.

7. The lateral flow assay device for traumatic brain injury using time-resolved fluorescence analysis of claim 6, wherein
in the antibody bound with europium particles, the mouse-origin antibody is further added at a concentration of 3% to 12%, preferably 3% in the adsorption pad spray solution.

8. The lateral flow assay device for traumatic brain injury using time-resolved fluorescence analysis of claim 7, wherein
GFAP which is one of traumatic brain injury markers is detected with sensitivity of less than 100 pg/mL using the rabbit-origin antibody as the capture antibody.

9. A method for diagnosing traumatic brain injury using a lateral flow assay device using time-resolved fluorescence analysis according to claim 4, the method comprising the steps of:
preparing a blood sample containing a traumatic brain injury marker,
injecting the blood sample containing the traumatic brain injury marker into a sample pad,
forming a traumatic brain injury marker complex consisting of a capture antibody labeled with a specific binding material and a detector antibody labeled with a fluorescent material while migrating the blood sample containing the traumatic brain injury marker along an adsorption pad adjacent to the sample pad with a capillary phenomenon,
migrating the traumatic brain injury marker complex along a porous film fluid-communicating with the adsorption pad to bind to a capture material on a detection line of the porous film,
binding any probe not binding to the traumatic brain injury marker to a capture material of a control line through the detection line, and
measuring a concentration of the traumatic brain injury marker by irradiating light to the detection line and the control line from a time-resolved fluorescence tester and comparing fluorescent signals of the detection line and the control line to diagnose the traumatic brain injury.

10. The method of claim 9, comprising:
diagnosing the traumatic brain injury in combination with a Glasgow coma scale (GCS) (awake, language function, and exercise function).

11. The method of claim 9, wherein
biotin is used as the specific binding material, europium is used as the fluorescent material, and a mixture of a mouse-origin antibody and a rabbit-origin antibody is used as an antibody for the detector antibody.

12. The method of claim 11, wherein
in an antibody bound with europium particles, the content of the rabbit-origin antibody is at a concentration of 0.1% to less than 2%, preferably 1% in an adsorption pad spray solution.

13. The method of claim 12, wherein
in the antibody bound with europium particles, the mouse-origin antibody is further added at a concentration of 3% to 12%, preferably 3% in the adsorption pad spray solution.

14. The method of claim 13, wherein
GFAP which is one of traumatic brain injury markers is detected with sensitivity of less than 100 pg/mL using the rabbit-origin antibody as the capture antibody.
